(19) United States
(12) Patent Application Publication
(10) Pub. No.: US 2015/0230770 A1
(43) Pub. Date: Aug. 20, 2015

(54) DIAGNOSIS STATION FOR DIAGNOSING MAMMOGRAMS

(71) Applicant: GENERAL ELECTRIC COMPANY, Schenectady, NY (US)

(72) Inventors: Peter Heinlein, Munchen (DE); Vanessa Del Valle Magalhaes, Munchen (DE); Axel Crasemann, Munchen (DE); Bruce SChroeder, Md, Greenville, NC (US)

(21) Appl. No.: 14/428,788

(22) PCT Filed: Sep. 17, 2013
(86) PCT No.: PCT/US2013/060084

§ 371 Date: Mar. 17, 2015

(30) Foreign Application Priority Data
Sep. 17, 2012 (DE) 102012216560.6
Nov. 19, 2012 (DE) 102012221118.7

(51) Int. Cl. A61B 6/00; A61B 6/03; A61B 6/02; G06F 3/041; G06F 3/0488

(52) U.S. Cl. CPC A61B 6/502; G06F 3/0416; G06F 3/0488; A61B 6/463; A61B 6/468; A61B 6/54; A61B 6/563; A61B 6/025; A61B 6/032

(57) ABSTRACT

A diagnosis station for diagnosing mammograms comprising a monitor device for displaying at least one mammogram, a computing device providing image data of the mammogram, and an input device for controlling the display of the mammogram. Herein, the input device comprises a touch sensitive touchscreen for controlling the display of the mammogram on the monitor device, the computing device provides first image data of the mammogram to the input device to display the mammogram on the touchscreen, the computing device provides second image data of the mammogram to the monitor device to display the mammogram on the monitor device, and the input device displays the mammogram according to the first image data on the touchscreen and, upon input of a control command by a user via the touchscreen, according to the mammogram displayed on the touchscreen, transfers control command data relating to the control command to the computing device.

### BACKGROUND

Embodiments of the present invention relate to a diagnosis station for diagnosing mammograms and to a method of controlling a diagnosis station for diagnosing mammograms.

A diagnosis station for diagnosing mammograms of this kind comprises a monitor device for displaying at least one mammogram, a computing device providing image data of the at least one mammogram to be displayed on the monitor device, and an input device for controlling the display of the at least one mammogram on the monitor device.

Conventionally, for diagnosing mammograms of a female patient taken at one or multiple X-ray breast examinations, a diagnosis workstation is employed comprising storage means for storing image data relating to the mammograms and comprising a high resolution monitor device for displaying a single mammogram or multiple mammograms at a time in a high resolution suitable for diagnosis. The mammograms herein are associated with a predefined so-called hanging protocol according to which mammograms of a patient taken in multiple examination sessions at multiple examination dates are arranged in a predefined order such that a physician, by stepping through the mammograms arranged according to the hanging protocol, may examine the mammograms and make a diagnosis based on for example a comparison of newly taken mammograms with older mammograms.

Within a hanging protocol, for example mammograms of the right and the left breast of a patient imaged in different orientations (for example in a so-called CC (craniocaudal) or a MLO (mediolateral oblique) orientation) are arranged side by side, such that a symmetry comparison between the left and the right breast is easily possible for a physician, which may facilitate identifying a possible diagnostic finding such as a tumour or a micro-calcification in a breast.

Typically, for stepping through the mammograms according to a hanging protocol and for displaying such mammograms on the monitor device, an input device is provided which is connected to the computing device and allows controlling the display of the mammograms on the monitor device in an easy and intuitive manner.

Conventionally, within mammography workstations, a dedicated keypad in addition to a keyboard and a mouse of the workstation is provided. The dedicated keypad herein is specifically provided for the mammography workstation and comprises a number of dedicated, specifically customized hardware keys which allow for example for a stepping from one mammogram to another, to zoom in and out of a mammogram, to invoke tools such as a local magnification or a spotlight tool to be applied to a mammogram or to mark a diagnostic finding within a mammogram.

Such dedicated keypad is typically provided because by means of the dedicated keys of the keypad a physician may in an easy and intuitive manner control the display of the mammograms on the monitor device. Because the keys on the keypad are arranged in an intuitive manner and because their number is limited, a physician can easily and quickly learn how to control the display of the mammograms by means of the keys, such that the control becomes easy and does not require the physician to look at the keypad while looking at the monitor device and for example stepping through mammograms arranged in a hanging protocol.

However, the use of a dedicated keypad has the disadvantage that new functions within a software for operating the diagnosing workstation cannot easily be integrated into the keypad, such that possibly an exchange of the keypad is necessary providing new hardware keys if new functions are to be implemented in a diagnosis software. This is costly and cumbersome.

In addition, typically the display of the mammographic data on the monitor device is not solely controlled by the dedicated keypad but in addition input commands must be entered via the mouse and the keyboard of the workstation. Hence, a physician must, for diagnosing mammograms, switch between the keypad, the mouse and the keyboard to control the display, enter a diagnostic finding and finalize a diagnosis of an examination case. Because the physician typically sits in a dark room when examining mammograms on the monitor device and because a physician typically examines a huge number of cases in a relatively short time (for example 100 cases per hour), the switching between the different control and input devices is distracting for the physician, hence complicating the diagnosis procedure for the physician.

There is a desire for an input device for a mammography workstation which allows for an easy and intuitive control of the display of mammographic data on a monitor device without the drawbacks of input devices currently in use.

### BRIEF SUMMARY OF THE INVENTION

It is an object of the invention to provide a diagnosis station for diagnosing mammograms and a method for controlling a diagnosis station for diagnosing mammograms which allow a physician to easily and intuitively control the display of mammograms on a monitor device, hence making a diagnosis procedure more comfortable and efficient.

This object is achieved by means of a diagnosis station for diagnosing mammograms according to embodiments of the present invention.

Accordingly, the input device comprises a touch sensitive touchscreen, wherein for controlling the display of the at least one mammogram on the monitor device the diagnosis station is constituted such that

the computing device provides first image data of at least one mammogram to the input device to display the at least one mammogram on the touchscreen,

the computing device provides second image data of the at least one mammogram to the monitor device to display the at least one mammogram on the monitor device, and

the input device displays the at least one mammogram according to the first image data on the touchscreen and, upon input of a control command by a user via the touchscreen according to the at least one mammogram displayed on the touchscreen, transfers control command data relating to the control command to the computing device.

The instant invention starts from the idea to use, as an input device, a touch device comprising a touch sensitive touchscreen. Via the touchscreen a command may be entered by a user through touching the touchscreen by tapping on or swiping across the touchscreen or by any suitable touching action relating to a control demand. The input device may be constituted as a so-called tablet computer or by any other device comprising a touch input in the shape of a touchscreen such as a smart phone, a music player or the like.

By using an input device comprising a touch input in the shape of a touch sensitive touchscreen, the need for a dedicated keypad is alleviated. Furthermore, also typical keyboard and mouse functions may be integrated into the touch input device such that a control of the display of the mammograms on the monitor device can solely be achieved by means of the input device, hence allowing for a control by a single input device.

During a diagnosis session, a physician should examine the mammograms displayed on the monitory device. It herein is essential that the physician is not distracted from the monitor device, and if possible, can control the display on the monitor device without moving his eyes away from the monitor device. (If a physician would have to look constantly back and forth between the monitor device and the input device, this would be tiring for the physician deteriorating his concentration.)

For this reason, the touch input device does not simply emulate keys which otherwise would have been used on a dedicated keypad but is provided with image data from the computing device to display the mammograms on the touchscreen which also are displayed on the monitor device. Hence, on the touchscreen similar mammographic data as on the monitor device are shown, and a user may in an intuitive manner enter control commands according to the mammograms displayed on the touchscreen, for example by tapping on a mammogram or by swiping across a mammogram.

In this regard, the first image data provided to the input device and the second image data provided to the monitor device relate to the same mammograms such that similar mammographic data are displayed on the touchscreen of the input device and on the monitor device. However, the monitor device typically is constituted by a specific, dedicated diagnosis monitor having a large number of pixels (for example five Megapixel per screen, wherein multiple screens may be used), whereas the touchscreen of the input device in comparison to the monitor device has a lower number of pixels. Hence, the monitor device may display the mammograms in a higher resolution than the touchscreen of the input device, such that the first image data provided by the computing device to the input device correspond to mammograms in a low resolution as compared to the second image data provided by the computing device to the monitor device corresponding to the mammograms in a high resolution.

Because the mammographic data are displayed on the input device in a decreased resolution as compared to the monitor device, a physician shall diagnose mammograms on the monitor device providing the mammograms in full resolution. The input device is solely used for controlling the display on the monitor device, but should not by itself be used for diagnosis.

In addition or alternatively, also the geometry, for example the aspect ratio, of displayed mammograms may vary between the monitor device and the touchscreen of the input device. In such case, standard strategies to fit the image with minimal cut-off into different viewport geometries of the monitor device and the touchscreen of the input device can be applied. For example, viewports of the monitor device and viewports of the input device may be different in size. Correlated mammographic images hence are shown both on the monitor device and the input device, wherein the exact shape of the displayed mammograms or the displayed section of the mammograms may be different according to the specific geometric viewport shape on the monitor device and on the input device.

The computing device represents a central device for storing image data and for providing image data to both the input device and the monitor device. First image data herein flow from the computing device to the input device, for example corresponding to low resolution image data of mammograms. Second image data flow from the computing device to the monitor device, for example corresponding to high resolution image data of the same mammograms also displayed on the touchscreen.

In addition, control command data flow from the input device to the computing device for controlling the display of mammograms on the monitor device. Such control command data, dependent on their nature, cause the computing device to provide image data to the input device and the monitor device to, for example, display a new set of mammograms on the touchscreen of the input device and on the monitor device.

Hence, in one embodiment no image data flows from the input device to the computing device, but only control command data. Image data is solely stored on a storage means of the computing device and provided from the computing device to both the input device and the monitor device. (This is in contrast to a nowadays typical application of for example a tablet computer in which data relating to for example video images are streamed from the tablet to another device such as a television, which not takes place in the instant case in which the streaming of image data originates from the computing device but not from the input device which only serves for controlling the display on the monitor device.)

The monitor device according to an embodiment is connected to the computing device—constituted for example as a workstation—by a cable as it is conventionally done for monitors. The input device and the computing device, however, communicate wirelessly in an embodiment using for example the WLAN technology or any other wireless communication standard such as Bluetooth.

The touchscreen may be embodied by any suitable touch sensitive display device. For example, a display with a capacitive touch sensor unit may be used.

In one embodiment, the monitor device, for displaying multiple mammograms, comprises multiple viewports, which represent place holders for displaying the mammograms in a predefined arrangement. Such viewports may be implemented by software and have the shape of rectangular boxes in which mammograms may be arranged on a screen for example side by side or in a matrix fashion in a predefined manner. Such viewports are, in an embodiment, mirrored on the touchscreen of the input device for displaying the multiple mammograms on the touchscreen in an arrangement corresponding to the arrangement on the monitor device, wherein the viewports displayed on the monitor device and the viewports displayed on the touchscreen of the input device in principle are independent from one another and may vary in size and aspect ratio. The touchscreen hence shows the mammograms displayed on the monitor device in the same order and arrangement such that, from their contents, the same mammograms are shown on the monitor device and on the touchscreen (as mentioned above, the mammograms displayed on the monitor device may have a higher resolution than the mammograms displayed on the touchscreen). Hence, a physician will see the same mammograms independent on whether he looks at the monitor device or the touchscreen. This makes it possible for a physician to see by a quick glance on the touchscreen where he may touch to enter an input command relating to a certain mammogram, and after once having located a mammogram on the touchscreen, he may not have to look at the touch input device at all any more but may move across the touchscreen from one mammogram to another as he sees the mammograms on the monitor device.

For controlling the display of the mammograms on the monitor device, touching actions may be employed which are nowadays also known from controlling for example a tablet computer or a smart phone employing a touchscreen input device. Within embodiments of the instant inventions such touching actions are, however, specifically customized for displaying mammograms and for controlling a monitor device of a mammography workstation.

For example, a tap by user on a mammogram displayed on the touchscreen may cause a control command for displaying another mammogram or another set of mammograms. Herein, a tap by user on a first portion of the touchscreen may cause a control command for displaying one or multiple mammograms according to a next step of a predefined hanging protocol, whereas a tap on a different, second portion of the touchscreen may cause a control command for displaying at least one mammogram according to a previous step of the predefined hanging protocol.

For example, by touching a right half of the touchscreen, a user may move to the next step of the hanging protocol. By touching a left half of the touchscreen, in contrast, it may be moved to a previous step of the hanging protocol.

Within the hanging protocol, mammograms relating to one or multiple mammography examinations of a patient are arranged in a predefined order such that, by stepping through the hanging protocol and the mammograms arranged therein, a physician may step through the mammograms relating to the patient in an order defined by the hanging protocol. Such hanging protocol may be configurable by the physician such that the physician himself may define in which order he wishes to examine mammograms relating to a patient.

For example, in a hanging protocol it may be defined that in a first step mammograms relating to a MLO orientation of a new and a previous examination of a left and a right breast shall be displayed on the monitor device and the touchscreen side by side. In a next step, mammograms relating to a MLO orientation of the left and the right breast as well as mammograms relating to a CC orientation of the left and the right mammogram shall be displayed side by side, and in a further step mammograms relating to a CC orientation of the left and right breast of a new examination and a previous, older examination shall be displayed side by side on the monitor device and the touchscreen. By tapping on the different portions of the touchscreen, the physician may move between such steps of the hanging protocol to move between different sets of mammograms to allow for a diagnosis of the mammograms by comparing the different mammograms.

An example of a method for managing mammographic data by means of a hanging protocol is for example described in EP 1 585 049 A2, which shall be incorporated herein by reference.

The monitor device, in an embodiment, displays only the mammograms themselves such that a physician on the monitor device may study the mammograms without being distracted by any other information or toolbar data. On the touchscreen, however, in addition to the mammograms further data relating to the control of the display may be shown. For example, above the mammograms displayed on the touchscreen, an image strip may be displayed indicating steps of a predefined hanging protocol for displaying mammograms in a predefined order. By means of the image strip, a user may move between the steps of the hanging protocol for example by swiping across the image strip and by thus moving the image strip from left to right or vice versa. A step of the hanging protocol may then be selected for display for example by tapping on a field relating to a step of the hanging protocol, such that the step of the hanging protocol is invoked and the mammograms relating to that step of the hanging protocol are displayed on the monitor device and the touchscreen.

A user may also move between mammograms by swiping across a mammogram displayed on the touchscreen.

For example, through swiping along a first direction, for example from left to right, across a mammogram displayed on the touchscreen a control command may be generated to navigate between like mammograms differing in their examination date (for example mammograms displaying the right breast of a patient in a CC orientation taken at different examination dates) such that it can be moved between older and newer mammograms.

By swiping along a different, second direction across a mammogram displayed on the touchscreen, for example from top to bottom, a control command may be generated to navigate through like mammograms of a single examination date. This is based on the fact that during a single examination date very often multiple images of the same breast of a patient in the same orientation are taken, such that duplicates of mammograms of a certain orientation for a single examination date are present. By swiping along the second direction across a mammogram, it can then be moved between such duplicate mammograms.

As another control command, a user may touch a mammogram displayed on the touchscreen at two touch points and may vary the distance between such touch points. If for example, two fingers are moved apart from another on a mammogram displayed on the touchscreen, it may be zoomed into a mammogram such that a mammogram or at least a portion of the mammogram is displayed in a larger size. If two fingers are moved to approach each other, in contrast, it may be zoomed out of a mammogram, hence decreasing the size of the mammogram.

As another control command, a user may touch a mammogram displayed on the touchscreen at a single touch point and may move the touch point to cause a control command to pan the associated mammogram.

In a specific embodiment, the computing device is constituted to transfer the control command data relating to the control command to the monitor device, wherein the monitor device is constituted to display the at least one mammogram relating to the second image data stored on the monitor device as commanded by the control command data. Hence, the monitor device receives image data from the computing device and stores it in a local memory. Then, if via the input device a command to alter the display of a currently displayed mammogram (for example a zoom command or a pan command) is entered, no new image data is provided to the monitor device, but rather the control command data is transferred to the monitor device and the monitor device adjusts the display of the mammograms accordingly. If in each case new image data were provided to the monitor device by the computing device, the displaying of the data in the monitor device would be deteriorated by a possibly limited data transfer speed between the computing device and the monitor device. If for example a user swipes fast across a mammogram in order to move a mammogram, the displaying on the monitor device would possibly be bumpy and jerky. If however only control command data are transferred to the monitor device and the monitor device then adjust the display by itself according to the received control command data, limitations in data transfer speed can be alleviated since the control command data generally comprise only a small data volume.

Possibly, all image data relating to a patient may be transferred to the monitor device and/or the input device at the beginning of a diagnosing examination, and the change of display of the mammograms on the monitor device then is changed according to control commands entered via the input device making use of the image data already stored on the monitor device and the input device. Hence, during an ongoing examination of a patient, no or very limited additional image data flow from the computing device to the monitor device and/or the input device.

In this way for example all image data relating to a stack of mammographic images as for example obtained from a tomosynthesis or a computer-tomography (CT) examination can be transferred to the monitor device and/or the input device at the beginning of an examination such that the image data are present at the monitor device and/or the input device during the examination and can quickly be stepped through without the need for additional image data transfer.

In addition, image data relating to another patient may already be transferred to the monitor device and/or the input device prior to initiating the examination of such patient. For example, during the examination of a patient image data relating to a next patient may already be transferred to the monitor device and/or the input device. In this way, image data relating to another (next) patient are already present at the monitor device and/or the input device prior to starting an examination of that particular patient case, such that it can be quickly switched to the examination of another patient case and for example image data relating to the other patient can beforehand be shown in an hanging protocol overview on the input device.

Further, toolbars may be displayed on the touchscreen of the input device which are not displayed on the monitor device. Such toolbars may be invoked only if they suitably may be applied. Hence, a toolbar is displayed only if it possibly is needed but it is not displayed on the touchscreen if it cannot be reasonably used. Such toolbar may, for example, allow for invoking tools such as a magnification tool, a spotlight tool, a reporting tool or any other suitable tool.

In one embodiment, upon invoking a marking tool on the input device the drawing of a marking shape on the touchscreen of the input device causes a control command to display a corresponding marking shape on the monitor device, wherein the monitor device is constituted to display additional information relating the marking shape. For example, a user may draw a circle on the touchscreen of the input device by swiping across the touchscreen. A corresponding circle, possibly adapted to an ideal shape, is then displayed also on the monitor device, wherein possibly further information such as arrows, distances to structural features, area sizes or the like are displayed on the monitor device. Because the additional information are displayed only on the monitor device, the attention of a user is directed to the monitor device and not to the input device.

The object is furthermore achieved by a method of controlling a diagnosis station for diagnosing mammograms in which:

a monitor device displays at least one mammogram,

a computing device provides image data of at least one mammogram to be displayed on the monitor device, and

an input device controls the display of the at least one mammogram on the monitor device.

Herein, the input device comprises a touch sensitive touchscreen, wherein for controlling the display of the at least one mammogram on the monitor device

the computing device provides first image data of the at least one mammogram to the input device to display the at least one mammogram on the touchscreen,

the computing device provides second image data of the at least one mammogram to the monitor device to display the at least one mammogram on the monitor device, and

the input device displays the at least one mammogram according to the first image data on the touchscreen and, upon input of a control command by a user via the touchscreen according to the at least one mammogram displayed on the touchscreen, transfers control command data relating to the control command to the computing device.

### BRIEF DESCRIPTION OF THE DRAWINGS

The advantages and the advantageous embodiments described above for the diagnosis station are equally applicable also for the method, such that it shall be referred to the above.

The idea underlying the invention shall subsequently be described in more detail with regard to the embodiments shown in the figures. Herein,

FIG. 1 shows a schematic view of a diagnosis station comprising a monitor device and an input device;

FIG. 2 shows a schematic view of a diagnosis station, indicating a data flow between a computing device, a monitor device and an input device;

FIG. 3 shows a schematic view of an input device;

FIG. 4 shows a touchscreen of an input device;

FIG. 5 shows another view of the input device;

FIG. 6 shows another view of the input device;

FIG. 7 shows another view of the input device together with a corresponding display of the monitor device;

FIG. 8 shows another view of the input device together with a corresponding display of the monitor device;

FIG. 9 shows another view of the input device together with a corresponding display of the monitor device;

FIG. 10 shows another view of the input device together with a corresponding display of the monitor device;

FIG. 11 shows another view of the input device together with a corresponding display of the monitor device;

FIG. 12A shows a touch input action of a user on a touchscreen of the input device;

FIG. 12B shows a mark on the touchscreen derived from the touch input; and

FIG. 12C shows a mark displayed on the monitor device in accordance with the mark derived from the touch input command.

### DETAILED DESCRIPTION

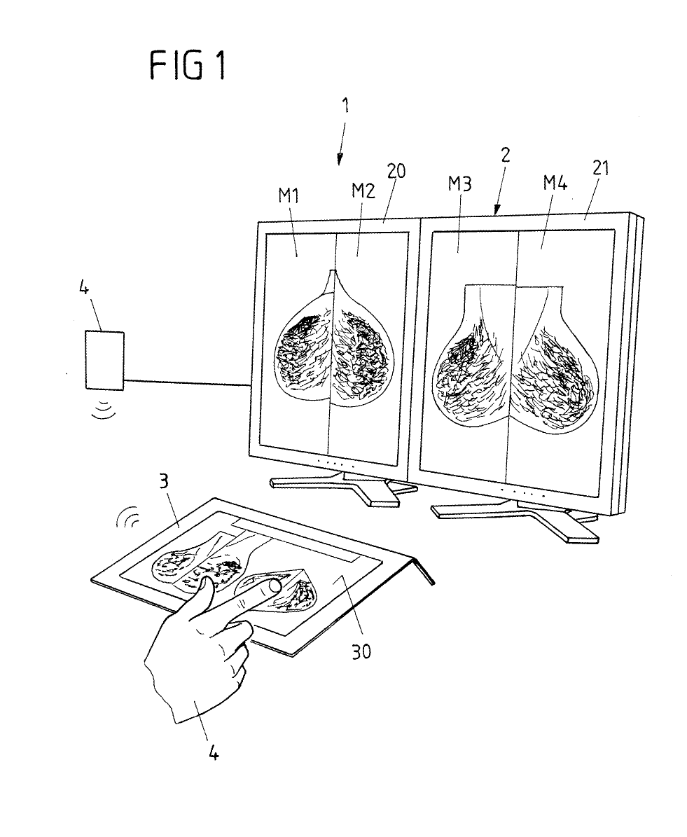

FIG. 1 shows a diagnosis station 1 comprising a computing device 4 in the shape of a workstation connected to a monitor device 2 comprising two screens 20, 21 for displaying a number of mammograms M1-M4. An input device 3 comprising a touch sensitive touchscreen 30 is provided which communicates wirelessly with the computing device 2.

The setup as it is shown in FIG. 1 is a typical arrangement as experienced by a physician for example in a hospital environment. The physician, for diagnosing mammograms, herein sits in front of the monitor device 2 and studies the mammograms M1-M4 displayed on the monitor device 2. The physician herein may step through the mammograms M1-M4 relating to a patient to reach a diagnosis and upon finalizing the diagnosis for the respective patient case may move to another patient for diagnosing mammograms M1-M4 relating to that patient.

A user U (typically a physician) may control the display of the mammograms M1-M4 on the monitor device 2 by means of the input device 3 which is constituted as a touch input comprising a touch sensitive touchscreen 30 via which the physician, by touching the touchscreen 30, may enter an input command, may navigate through mammograms M1-M4, may move to a next patient or may identify and report a diagnostic finding.

The touch input device 3 may have the shape of a tablet computer and may comprise a capacitive touch sensor which capacitively sensors a touch of the touchscreen 30 by a user U. In response to the touch, control command data is generated which is provided to the computing device 4 for controlling the display of the mammograms M1-M4 on the monitor device 2.

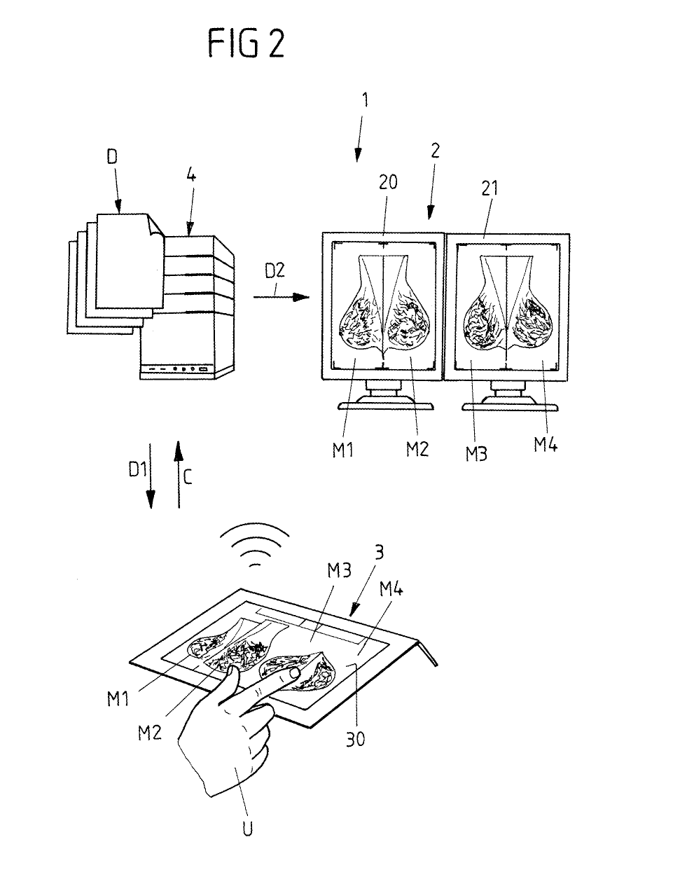

FIG. 2 shows in a schematic view the data flow between the computing device 4, the monitor device 2 and the input device 3.

The computing device 4 comprises storage means for storing image data D relating to mammography examinations. If mammograms M1-M4 are to be displayed on the screens 20, 21 of the monitor device 2, image data D2 is provided from the computing device 4 to the monitor device 2, and the corresponding mammograms M1-M4 are displayed on the monitor device 2. Such image data D2 typically represent high resolution data such that the mammograms M1-M4 are displayed on the monitor device 2 in a high resolution exploiting the high resolution of the screens 20, 21 of the monitor device 2 (each screen 20, 21 may for example comprise five Megapixels). The mammograms M1-M4 are hence displayed on the monitor device 2 in full resolution allowing for a diagnosis of the mammograms M1-M4.

The computing device 4 also provides image data D1 to the input device 3, wherein the image data D1 relates to the same mammograms M1-M4 which are also displayed on the monitor device 2. According to the image data D1, the mammograms M1-M4 are displayed on the touchscreen 30 of the input device 3, however in a lower, reduced resolution as compared to the screen 20, 21 of the monitor device 2. Hence, the image data D1 are reduced in size compared to the image data D2 provided to the monitor device 2.

A user U may input a control command by touching the touchscreen 30 in a region in which a mammogram M1-M4 is displayed on the touchscreen 30. By touching the touchscreen 30 control command data C are generated, which are transferred from the input device 3 to the computing device 4. According to the control command data C, the computing device 4 provides updated image data D1, D2 to the monitor device 2 on the one hand and to the input device 3 on the other hand for modifying the display of the mammograms M1-M4 or for displaying other mammograms M1-M4 for example by selecting a new patient case or another set of mammograms relating to the same patient or the like.

According to the newly provided image data D1, D2, the monitor device 2 and the input device 3 display the mammograms M1-M4 in a modified fashion or display other mammograms M1-M4.

The computing device 4 hence represents a central device for storing and providing image data D, D1, D2. Such image data D, D1, D2 are streamed to both the monitor device 2 and the touchscreen 3. In return, the input device 3 sends control command data C to the computing device 4 which are processed by the computing device 4 and cause further image data D1, D2 to flow from the computing device 4 to the monitor device 2 and the input device 3. Herein, no image data are streamed from the input device 3 to the computing device 4 or the monitor device 2.

Via the input device 3 a user U may control the display of the mammograms M1-M4 on the monitor device 2, wherein the input device 3 communicates wirelessly with the computing device, for example via a wireless local area network (WLAN) or via any other suitable communication standard such as Bluetooth.

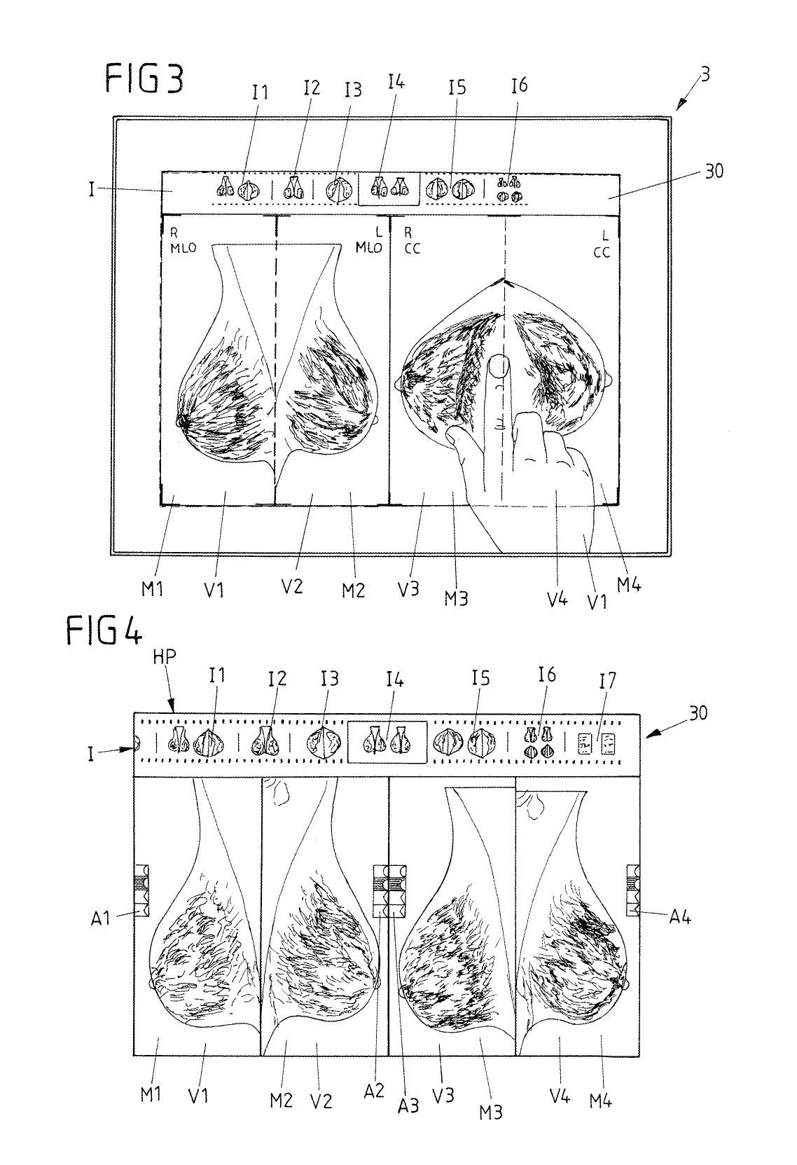

Subsequently, according to FIGS. 3 to 12A-12C, it shall be described how certain touch actions performed by a user U on the touchscreen 30 may be used to control the display of the mammograms M1-M4 on the monitor device 2.

As shown in FIG. 3, mammograms M1-M4 are shown on the touchscreen 30 of the input device 3 in so-called viewports V1-V4 representing place holders in the shape of rectangular boxes for displaying one mammogram M1-M4 each. Such viewports V1-V4 are equally present on the screens 20, 21 of the monitor device 2 and on the touchscreen 30, such that the same mammograms M1-M4 are shown in a corresponding viewport arrangement both on the monitor device 2 and on the input device 3.

The monitor device 2, in an embodiment, shows only the mammograms M1-M4, but no toolbars or other data relating to the control of the display of the mammograms M1-M4. On the touchscreen 30, however, as shown for example in FIGS. 3 and 4, in addition to the mammograms M1-M4 toolbars or other information data may be shown allowing for invoking tools for modifying the display of the mammograms M1-M4 or for stepping through certain arrangements of mammograms for comparing different mammograms M1-M4 relating to a patient.

The mammograms M1-M4 relating to a patient may be arranged for example according to a predefined so-called hanging protocol HP in which the mammograms are arranged in certain sets in certain configurations such that sets of mammograms M1-M4 can be stepped through in a predefined order. Such steps I1-I6 of the hanging protocol HP are indicated in an image strip I above the mammograms M1-M4 displayed on the touchscreen 30. Such steps I1-I6 may include

an arrangement of mammograms relating to a previous examination of the left and right breast of a patient in different orientations (step I1),

an arrangement of mammograms relating to a new examination of the left and right breast of the patient in a MLO orientation (step I2),

an arrangement of mammograms of a new examination of the left and right breast of the patient in a CC orientation (step I3),

a comparison of the MLO mammograms of the previous examination and the new examination of the patient (step I4),

a comparison of the CC mammograms of the previous examination and the new examination of the patient (step I5), and

a comparison of the MLO and the CC mammograms of the new and the previous examination of the patient (step I6),

wherein further and other steps may be defined in a hanging protocol HP.

The mammograms M1-M4 according to a currently selected step I4 of the hanging protocol HP are displayed on the touchscreen 30 of the input device 3 and at the same time on the monitor device 2. A user U may move to a next step of the hanging protocol HP (in this case step I5) by tapping on a right half of the touchscreen 30 (corresponding to the region of viewports V3 and V4) and may step to a previous step (in this case step I3) by tapping on a left half of the touchscreen 30 (corresponding to the region of viewports V1, V2). Hence, by a single tap a user U may navigate to another step of the hanging protocol HP in a sequential order, wherein upon a tap corresponding control command data C is generated and provided to the computing device 4, wherein the computing device 4 provides the corresponding image data D1, D2 in high resolution to the monitor device 2 and in low resolution to the input device 3 for displaying the mammograms M1-M4 relating to the selected other step of the hanging protocol HP.

Because the area of the region a user U must tap on for stepping to another step of the hanging protocol HP is large, no special attention must be spent by the user U on the touchscreen 30, but the user U may direct his full attention to the monitor device 2 and the mammograms M1-M4 displayed thereon, such that an easy, intuitive stepping through the mammograms M1-M4 with minimum distraction of a user U is achieved.

A user U may also swipe across the image strip I to move the image strip I to the left or to the right and may then select a certain step I1-I6 of the hanging protocol HP as indicated in the image strip I by tapping on the respective step I1-I6. Hence, by means of the image strip I, a certain step of the hanging protocol HP may be selected in a non-sequential manner, thus allowing for a direct access to a certain step I1-I6 of the hanging protocol HP without having to step through the hanging protocol HP until reaching the respective step I1-I6.

As also shown in FIG. 4, icon lists A1-A4 are displayed in respect of the mammograms M1-M4 on the touchscreen 30. Such icon list A1-A4 serve to indicate what version of a mammogram M1-M4 currently is displayed in the respective viewport V1-V4. Via the icon list A1-A4, it can, for example, be indicated how the currently displayed mammogram M1-M4 relates to other mammograms M1-M4 of the same patient, the same orientation (MLO, CC) and the same breast on a timeline such that a user U can obtain, from the icon list A1-A4, information with regard to whether currently an old mammogram M1-M4 or a new mammogram M1-M4 is displayed.

To navigate through old and new mammograms of the same patient, the same orientation (MLO, CC) and the same breast, a user U can for example, as shown in FIG. 5, swipe across a certain mammogram M3 from left to right or vice versa (along direction X). Through the swiping a user U may move from one mammogram M3 to another mammogram M3 relating to the same patient and the same breast and the same orientation, but taken at another examination date.

Further, as indicated in FIG. 5, a user U may swipe along a direction Y in an upwards or downwards direction for moving between duplicates of a mammogram M3 relating to the same patient and the same breast and the same orientation and also the same examination date. This is based on the fact that multiple mammograms M3 of the same breast of a patient in the same orientation may be taken at a single examination date, such that multiple like mammograms M3 are present for a single examination date. To move between such like mammograms M3, the user U may swipe across a mammogram M3 and hence may move from one mammogram M3 to another duplicate of that mammogram M3.

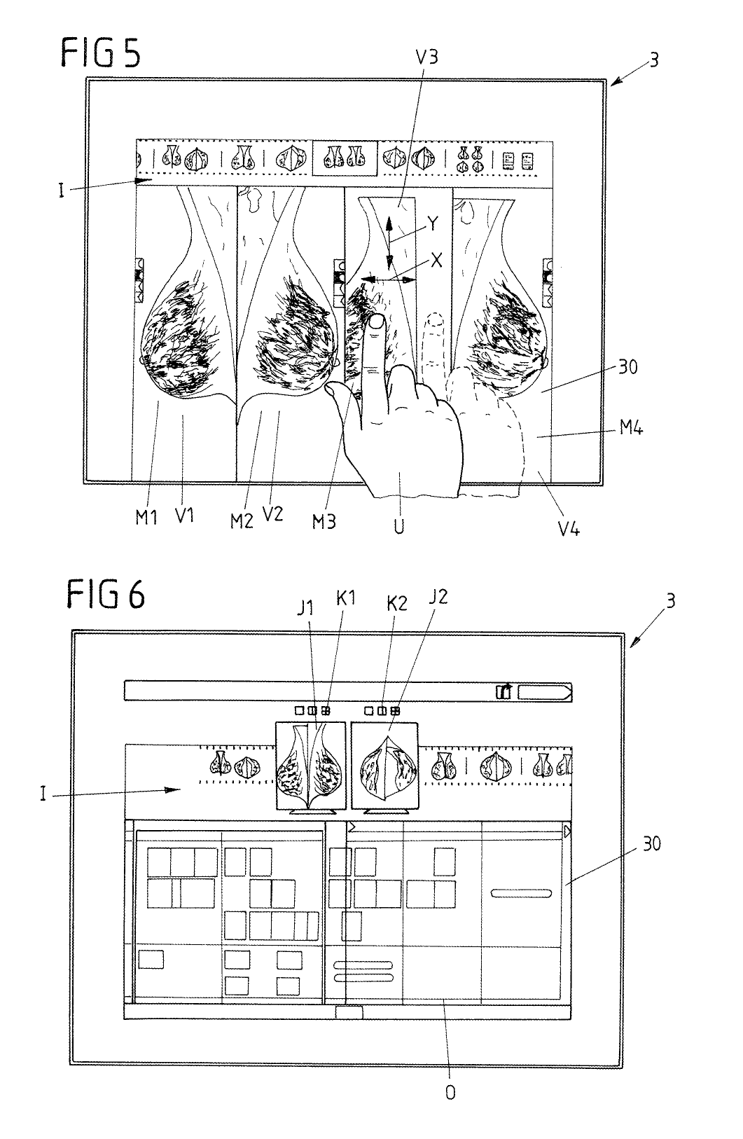

FIG. 6 shows another view displayed on the touchscreen 3 indicating the arrangement of mammograms M1-M4 in a case overview O displaying all available mammograms relating to a patient.

The view of FIG. 6 in particular allows for defining steps I1-I6 of a hanging protocol HP by selecting mammograms out of the case overview O and by moving them into fields J1, J2 allowing for an arrangement of mammograms M1-M4 according to a preselected arrangement K1, K2. Hence, a physician may configure steps I1-I6 of a hanging protocol HP according to his personal wishes using the case overview O as displayed on the touchscreen 30 as shown FIG. 6.

The case overview O as shown in FIG. 6 is, for example, shown only on the input device 3, but not on the monitor device 2, such that the physician may use the input device 3 to configure steps I1-I6 of a hanging protocol HP for later on examining different patient cases.

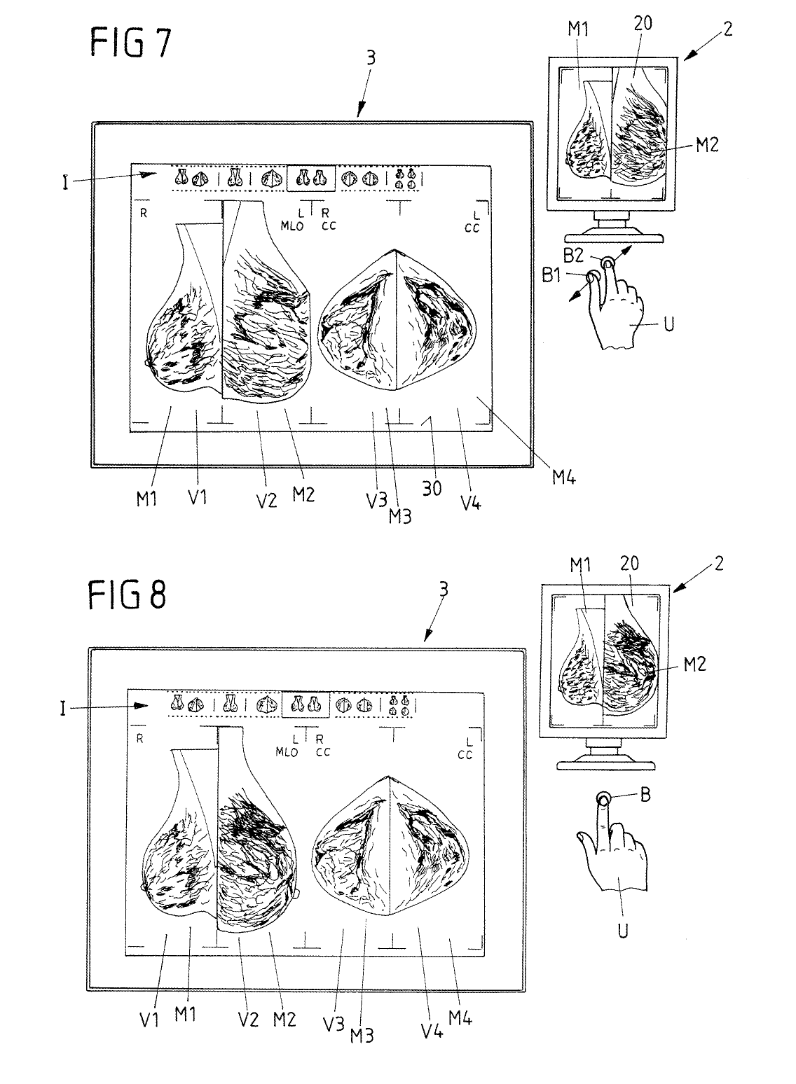

Referring now to FIG. 7, by using the input device 3, a user may also zoom in or zoom out of an image. For this, a user U may touch the touchscreen 30 at two touch points B1, B2, and by moving the touch points B1, B2 apart from one another a mammogram (in this case mammogram M2) is enlarged. The user U hence touches the mammogram M2 in question and moves his fingers (as he is used to from conventional operation of tablet computers and smart phones) and in this way may zoom in a mammogram M2 by moving his fingers apart from one another or may zoom out of a mammogram M2 by moving his fingers to approach each other. The zooming of the mammogram M2 is equally displayed on the monitor device 2.

Furthermore, as shown in FIG. 8, a user U may touch a mammogram (in this case mammogram M2) at a single touch point B, and by moving the touch point B he may pan the corresponding mammogram M2, which equally is also displayed on the monitor device 2.

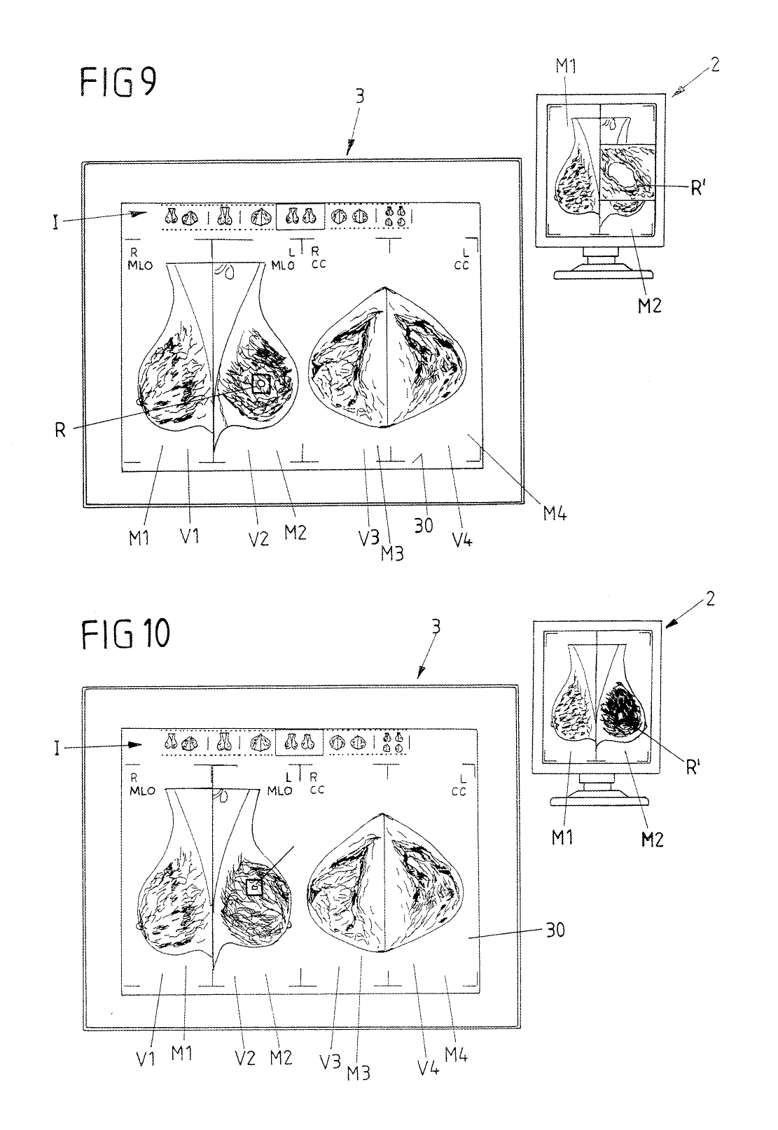

In addition, referring now to FIG. 9, a user U may invoke a magnifying tool. Once the magnifying tool is invoked, he may mark a region R on a mammogram M2 on the touchscreen 30 of the input device 3, which region R′ is then displayed in a magnified fashion on the monitor device 2.

Further, referring to FIG. 10, a user U may invoke a so-called spot light tool by which he locally can modify image characteristics, for example, by enhancing a contrast or brightness of structural features. Upon invoking the spotlight tool, a user U marks a region R in a mammogram M2 of the touchscreen of the input device 3, and within a corresponding region R′ structural features of a mammogram M2 are displayed in a modified fashion on the monitor device 2.

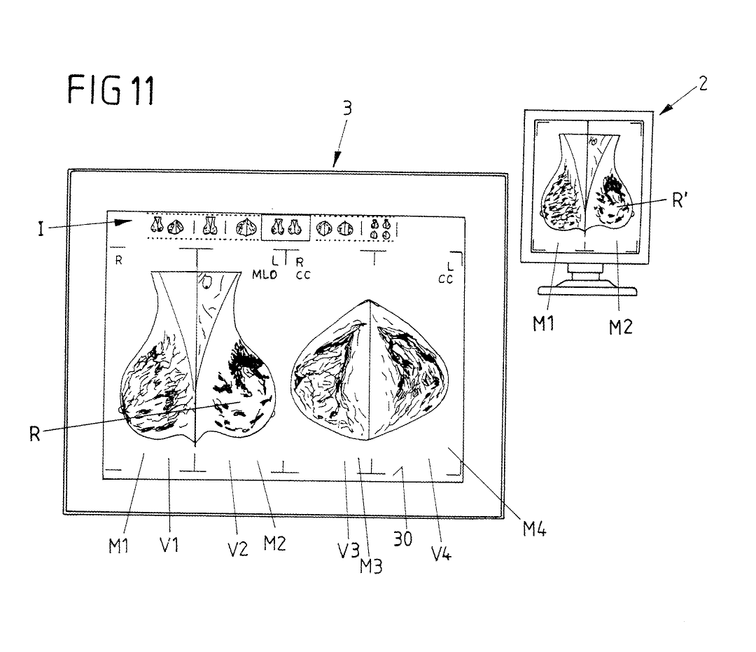

Further, referring now to FIG. 11, a user U may invoke a windowing tool. Once the windowing tool is invoked, a user may mark a window region R on a mammogram M2 on the touchscreen 30 of the input device 3, and correspondingly the mammogram M2 is windowed on the monitor device 2. By means of a windowing tool the brightness and/or image contrast may be adjusted in a certain region or throughout an entire mammographic image.

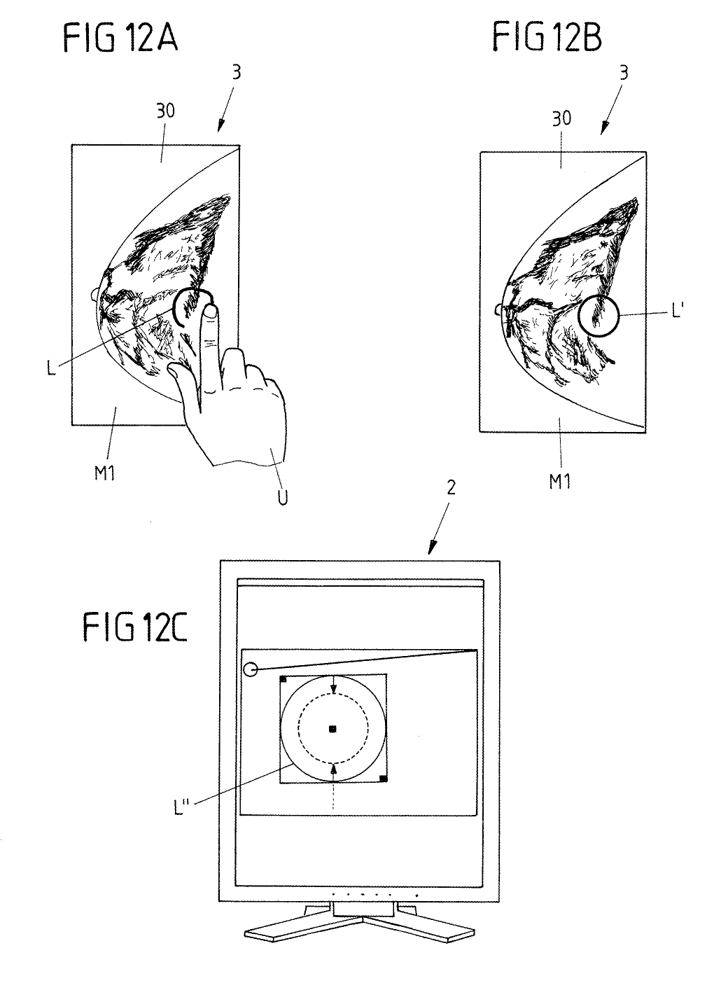

Further, referring now to FIG. 12A-12C, a user may invoke a marking tool allowing for a marking of regions of a mammogram M1. Once the marking tool is invoked, a user U may, for example, draw a line L in a mammogram M1 on the touchscreen 30 of the input device 3. From the—possibly buckled—line L drawn by the user U on the touchscreen 30, the input device 3 derives a smooth line L′ relating to, for example, a circular mark as shown in FIG. 12B, and correspondingly lines L″ relating to the mark are also displayed on the monitor device 2, as indicated in FIG. 12C. The lines L″ herein may include further details such as arrows and distances to structural features of a breast such as a breast nipple, which are automatically displayed to the user U on the monitor device 2, but not on the touchscreen 30 of the input device 3.

The idea underlying the invention is not limited to the embodiments described above, but may be realised also within completely different embodiments.

In particular, other touching actions may be defined to modify the display of mammograms on both the touchscreen of the input device and on the monitor device.

The input device is a touch input device, but in this regard may have any shape. The input device not necessarily is a tablet computer, but may also be a smart phone or any other suitable input device comprising a touchscreen.

The input device may also be physically connected to the computing device by means of a cable and does not necessarily have to communicate wirelessly with the computing device.

The monitor device may be any suitable device for displaying mammographic data and may also be, for example, a projector or the like.

1. A diagnosis station for diagnosing mammograms, comprising:
a monitor device configured to display at least one mammogram;
a computing device configured to provide image data of the at least one mammogram; and
an input device configured to control the display of the at least one mammogram on the monitor device, the input device comprising a touch sensitive touchscreen,
wherein:
the computing device provides first image data of the at least one mammogram to the input device to display the at least one mammogram on the touchscreen,
the computing device provides second image data of the at least one mammogram to the monitor device to display the at least one mammogram on the monitor device, and
the input device displays the at least one mammogram according to the first image data on the touchscreen and, upon input of a control command by a user via the touchscreen, according to the at least one mammogram displayed on the touchscreen, and transfers control command data relating to the control command to the computing device.

2. The diagnosis station according to claim 1, wherein the monitor device is further configured to display the at least one mammogram in a higher resolution and/or a different geometry than the touchscreen of the input device.

3. The diagnosis station according to claim 1, wherein the first image data provided by the computing device to the input device correspond to the at least one mammogram in a first resolution, whereas wherein the second image data provided by the computing device to the monitor device correspond to the at least one mammogram in a second resolution higher than the first resolution.

4. The diagnosis station according to claim 1, wherein the computing device and the input device communicate wirelessly.

5. The diagnosis station according to claim 1, wherein the monitor device comprises multiple viewports for displaying a plurality of mammograms in a predefined arrangement, wherein the viewports are mirrored independently on the touchscreen of the input device for displaying the plurality of mammograms on the touchscreen in a corresponding arrangement.

6. The diagnosis station according to claim 1, wherein a tap by a user on a mammogram displayed on the touchscreen causes a control command for displaying another mammogram or another set of mammograms.

7. The diagnosis station according to claim 1, wherein a tap by a user on a first portion of the touchscreen causes a control command for displaying at least one mammogram according to a next step of a predefined hanging protocol, and a tap on a second portion of the touchscreen causes a control command for displaying at least one mammogram according to a previous step of the predefined hanging protocol.

8. The diagnosis station according to claim 1, wherein the touchscreen, in addition to the at least one mammogram, displays an image strip indicating steps of a predefined hanging protocol for displaying mammograms in a predefined order.

9. The diagnosis station according to claim 8, wherein a swiping by a user across the image strip causes a control command for navigating between the steps of the predefined hanging protocol.

10. The diagnosis station according to claim 1, wherein a swiping by a user along a first direction across a mammogram displayed on the touchscreen causes a control command to navigate between like mammograms differing in their examination date.

11. The diagnosis station according to claim 10, wherein a swiping by the user along a second direction across the mammogram displayed on the touchscreen causes a control command to navigate between like mammograms of a single examination date.

12. The diagnosis station according to claim 1, wherein a touching of a mammogram displayed on the touchscreen at two touch points and a varying of the distance between the touch points causes a control command to vary the size of the mammogram.

13. The diagnosis station according to claim 1, wherein a touching of a mammogram displayed on the touchscreen at a single touch point and a moving of the touch point causes a control command to pan the mammogram.

14. The diagnosis station according to claim 1, wherein the computing device is constituted to transfer the control command data relating to the control command to the monitor device, wherein the monitor device is constituted to display the at least one mammogram relating to the second image data stored on the monitor device as commanded by the control command data.

15. The diagnosis station according to claim 1, wherein a toolbar is displayed on the touchscreen for a user to select a tool to be applied to a mammogram displayed on the touchscreen.

16. The diagnosis station according to claim 1, wherein, upon invoking a marking tool on the input device, the drawing of a marking shape on the touchscreen of the input device causes a control command to display a corresponding marking shape on the monitor device, wherein the monitor device is configured to display additional information relating the marking shape.

17. A method of controlling a diagnosis station for diagnosing mammograms, wherein the diagnosis station comprises a monitor device configured to display at least one mammogram, a computing device configured to provide image data of the at least one mammogram, and an input device configured to control the display of the at least one mammogram on the monitor device, wherein the input device comprises a touch sensitive touchscreen, the method comprising:
controlling the display of the at least one mammogram on the monitor device;
providing first image data of the at least one mammogram to the input device to display the at least one mammogram on the touchscreen;
providing second image data of the at least one mammogram to the monitor device to display the at least one mammogram on the monitor device;
displaying the at least one mammogram according to the first image data on the touchscreen and, upon input of a control command by a user via the touchscreen, according to the at least one mammogram displayed on the touchscreen; and
transferring control command data relating to the control command to the computing device.
